(19) United States
(12) Patent Application Publication
(10) Pub. No.: US 2008/0200952 A1
(43) Pub. Date: Aug. 21, 2008

(54) Bone Fixator

(75) Inventors: Christopher Ian Moorcroft, Staffordshire (GB); Peter Jan Ogrodnik, Staffordshire (GB); Peter Brian Macfarlane Thomas, Staffordshire (GB)

Correspondence Address: SHERIDAN ROSS PC, 1560 BROADWAY, SUITE 1200, DENVER, CO, 80202, US

(73) Assignee: INTELLIGENT ORTHOPAEDICS LTD, Stoke on Trent (GB)

(21) Appl. No.: 11/916,637

(22) PCT Filed: Jun. 7, 2006
(86) PCT No.: PCT/GB2006/002103

§ 371 Date: Apr. 8, 2008

(30) Foreign Application Priority Data
Jun. 13, 2005 (GB) 0511850.0

(51) Int. Cl. A61B 17/58

(52) U.S. Cl. 606/246; 606/96; 606/103

(57) ABSTRACT

Bone fixator for use in the treatment of a fractured bone comprising a support beam having means for attaching each end thereof to the respective ends of a fractured bone, the support beam being configured so as to permit predetermined relative movement between the respective ends of the support beam and thereby transmitting said relative movement to the respective ends of the fractured bone.

This invention relates to the field of external bone fixators for use in the treatment of fractured bones.

### BACKGROUND

Bone is capable of self-healing at a fracture site by the formation of callus which is able to reunite the ends of the fractured bone. Callus formation is triggered and maintained by relative movement of the fractured bone ends and occurs during a specific and limited time period following occurrence of the fracture.

If allowed to heal completely naturally, a fractured bone would heal in a poorly aligned condition, resulting in consequential future problems. Therefore the fractured bone ends are more usually manipulated into a well-aligned condition (fracture reduction) before callus formation and the natural healing process occurs. Once reduced, the fracture needs to be supported or fixed in order to maintain the desired alignment.

Rigid fixation of the fractured bone ends means that they are kept well aligned but may lead to a reduction or prevention of the formation of callus, therefore prolonging the natural healing process.

Treatment of a bone fracture by providing external support (e.g. a plaster of Paris cast) allows relative movement of the fractured bone ends to occur, which promotes callus formation. However, such external supports may not be suitable to assist with the need to accurately align the fractured bone ends, particularly with unstable or metastable fractures.

To alleviate these problems, external bone fixators have been developed which hold the fractured bone ends together sufficiently rigidly to maintain accurate alignment and yet at the same time allow sufficient relative movement between the fractured bone ends to promote callus formation. Such external fixators are applied externally to the injured limb and are attached to the fractured bone ends by bone pins or screws which pass through the soft tissue of the limb and into the bone.

Bridging the gap between the pins in the two ends of the fractured bone is a support mechanism, which holds the bone ends in alignment. To promote callus formation, the external fixator can be adapted to allow specific and controlled types of movement between the fractured bone ends. Such movement is generally effected by a corresponding movement in the fixator itself, e.g. relative axial movement between two component parts of the fixator can lead to corresponding relative axial movement of the fractured bone ends.

An example of an external fixator of this type is described in U.S. Pat. No. 5,320,622 (Orthofix Sri). Other examples of prior art fixators are described in EP1351613 (Mitkovic) and EP1434531 (Langmaid et al).

The prior art fixators are relatively complex, having numerous component parts and joints which are necessary in order to allow the fixator to provide selective retention of a wide variety of angular relations so that positional and angular adjustment can readily be applied to the fractured bone.

However, this complexity makes the fixator relatively expensive, heavy and bulky; the latter two factors being particularly undesirable from the point of view of a patient who may need to wear the fixator for many weeks.

Furthermore, most surgeons reduce fractures by manipulation, apply bone screws and the external fixator and then manipulate the fracture again to improve the reduction before locking the fixator. This second stage of reduction often results in the bone screws in the respective ends of the fractured bone no longer being longitudinally aligned, so that the fixator has to be locked in a position which is no longer in line with the longitudinal axis of the bone, resulting in unpredictable mechanical properties and movement. Furthermore, a consequence of the need to design joints capable of accommodating all potential eventualities may result in a final configuration of the device that is mechanically unsound.

It is therefore an object of the present invention to provide an external fixator which seeks to alleviate the problems of the above-described prior art.

### SUMMARY OF THE INVENTION

According to a first aspect of the present invention there is provided a bone fixator for use in the treatment of a fractured bone comprising a support beam having means for attaching each end thereof to the respective ends of a fractured bone, the support beam being configured so as to permit predetermined relative movement between the respective ends of the support beam and thereby transmitting said relative movement to the respective ends of the fractured bone.

Preferably, the support beam is a one-piece support beam with no articulated joints therein. This simplifies the manufacture of the fixator and reduces the complexity of its operation.

Preferably, the means for attaching each end of the support beam to the respective ends of a fractured bone comprise apertures for receiving therein bone pins, bone screws, wires or the like. The apertures can be very accurately positioned on the fixator so as to contribute to the predictable nature of the predetermined movement, reproducible from one fixator to another of the same type.

Preferably, the apertures can be used as location guides for the drilling of holes for the bone pins, bone screws, wires or the like.

Preferably, the fixator further comprises bone screw sleeves, each of which locate in one of said apertures, each bone screw sleeve having a bore therethrough for receiving therein bone pins, bone screws, wires or the like. If desired, one or more of said bone screw sleeves can have an angled bore therethrough in order to direction a bone screw to a particular location.

In a preferred form, at least one of said apertures can be used as a healing indicator by observing the possible positions of a bone pin or bone screw located therein relative to the periphery of said aperture.

Preferably said bone fixator is disposable i.e. for single-use. This is made feasible by the greatly simplified (and hence cheaper) construction of the fixator when compared with prior art fixators.

In a preferred form, the predetermined relative movement between the respective ends of the support beam is a property of the material of the support beam.

Alternatively, or in addition, the predetermined relative movement between the respective ends of the support beam is a property of the shape of the support beam.

Preferably, said support beam is made from a titanium alloy. Alternatively, said support beam is made from a layered composite material.

In one embodiment, the support beam includes one or more actuators which, in use, generate or contribute to said predetermined movement.

Preferably, said fixator is adapted to receive three bone pins, bone screws, wires or the like at each end thereof.

Preferably, the fixator is adapted to support a hybridisation component such as a T-bar attachment or ring attachment at one end thereof.

According to a second aspect of the invention, there is provided a bone fixation system comprising
a) a bone fixator as claimed in any of the preceding paragraphs; and
b) bone pins, bone screws, wires or the like for attaching the fixator to the respective ends of a fractured bone.

Preferably, the bone fixation system further comprises a hybridisation component such as a T-bar attachment or ring attachment.

Preferably, the bone fixation system further comprises comprising drilling guides for locating a drill with respect to said fixator.

### BRIEF DESCRIPTION OF THE DRAWINGS

Preferred embodiments of the present invention will now be more particularly described, by way of example only, with reference to the accompanying drawings wherein:

FIG. 1 is a perspective view of an external fixator embodying the first aspect of the present invention;

FIG. 1A is a side view of a bone screw locating sleeve;

FIG. 2 is a side view of the fixator with the bone screws and one bone screw locating sleeve in place;

FIG. 3 is a perspective view of the fixator of FIG. 2, with all bone screw locating sleeves in place;

FIG. 4 is a perspective view of the fixator of FIG. 1, with the bone screw locating sleeves in place;

FIG. 5 is a side view of the fixator of FIG. 4, indicating possible alternative cross-sectional shapes;

FIG. 6 is a top view of an alternative embodiment of the fixator which is adapted for use with a T-bar attachment;

FIGS. 6A-6C are further views of the T-bar attachment of FIG. 6, showing alternative configurations;

FIG. 7 is a side view of an alternative embodiment of the fixator which is adapted for use with a ring attachment;

FIG. 7A is a top view of the ring attachment of FIG. 7;

FIG. 8 is a side view of the fixator in use as a healing indicator;

FIG. 8A is a top view of a bone screw within an aperture in an unloaded condition; and

FIG. 8B is a top view of a bone screw within an aperture in a loaded condition.

### DETAILED DESCRIPTION

Throughout the description and claims of this specification, the words “comprise” and “contain” and variations of the words, for example “comprising” and “comprises”, means “including but not limited to”, and is not intended to (and does not) exclude other components, Integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith.

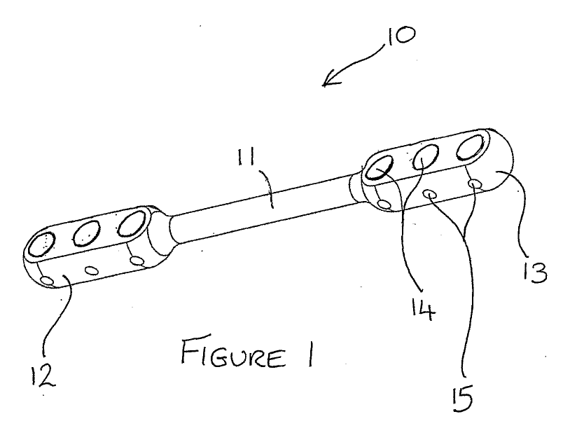

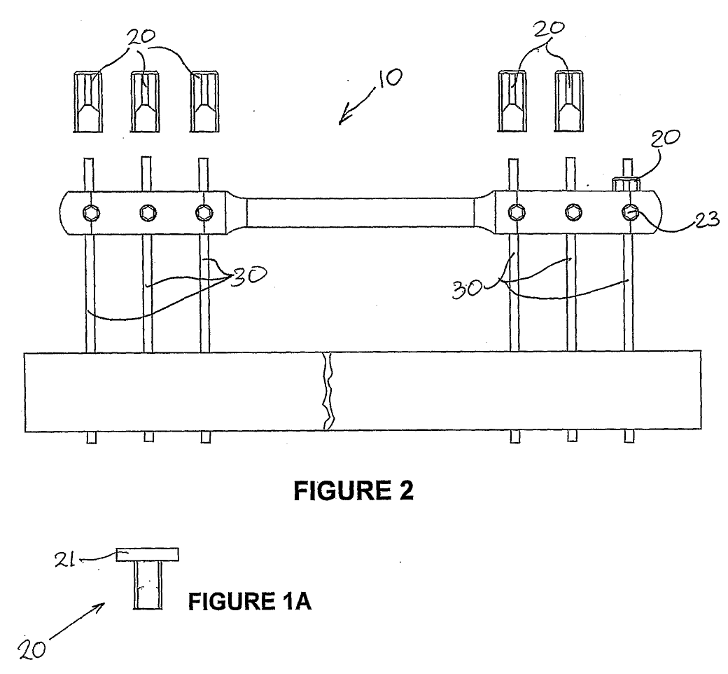

Referring to FIG. 1, the fixator 10 comprises an elongate support beam 11 having enlarged heads 12, 13 at either end thereof. The support beam is formed from one piece of a titanium alloy (for example) and has no joints, hinges or other articulation therein.

Each of the enlarged heads 12, 13 has three carefully positioned and aligned apertures 14 therethrough, which serve as the means for attaching the fixator 10 to the respective ends of a fractured bone.

Referring to FIG. 1A, bone screw locating sleeves 20 are provided, having a generally cylindrical shape and of suitable diameter to fit into one of the apertures 14. A flange 21 at the upper end of the bone screw locating sleeve 20 prevents it from falling through the aperture 14, in use. Each bone screw locating sleeve 20 and aperture 14 is provided with a lateral hole 22, 15. The lateral holes 22, 15 are aligned when the bone screw locating sleeve 20 is placed in an aperture 14 so that a fixing such as a grub screw or the like (not shown in FIGS. 1 or IA) can be inserted therein.

FIG. 2 shows the fixator 10, bone screw locating sleeves 20 and bone screws 30 partially assembled-together. An alternative embodiment of the bone screw locating sleeves 20 is shown in which no upper flange 21 is present. In FIG. 2, the rightmost bone screw locating sleeve is shown in place in the fixator, held in place by a grub screw 23. The exterior surfaces of the bone screw locating sleeves 20 are preferably not entirely in contact with the interior surface of apertures 14 and may have detents, grooves or other surface markings in order to reduce the adhesive effect of blood or general detritus at the interface between the sleeves 20 and apertures 14.

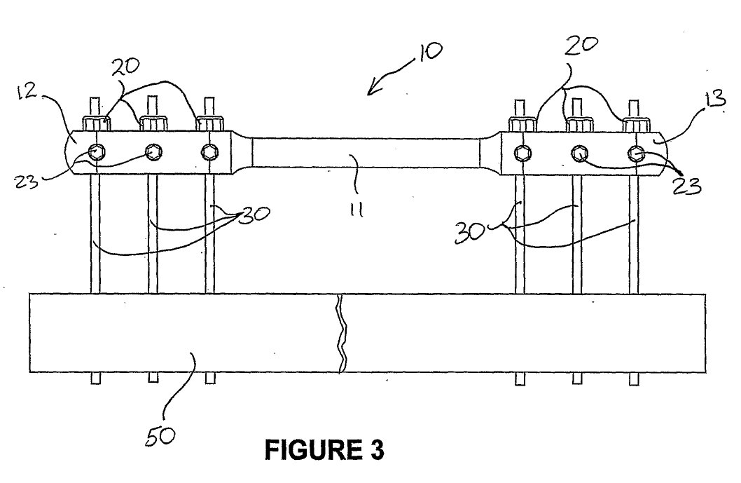

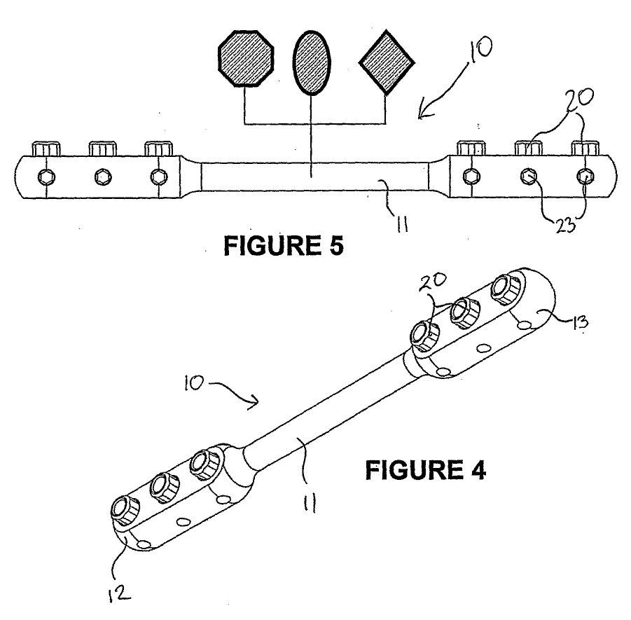

FIGS. 3 and 4 show the fixator 10 and bone screw locating sleeves 20 assembled together. FIG. 3 additionally shows the bone screws 30, fixings 23 and the fractured bone 50. As shown in FIG. 3, each aperture 14 contains a bone screw locating sleeve 20 and a bone screw 30. Fixings 23 fit into lateral holes 22, 15 to fix the bone screws 30 with respect to the fixator 10. Each set of three bone screws is fixed into the respective end of a fractured bone 50.

Since the bone screws 30 are rigidly fixed with respect to the bone screw locating sleeves 20 and enlarged heads 12, 13, relative movement between the respective ends of the fractured bone is only possible by means of corresponding movement of the support beam 11.

Movement of the support beam 11 is critical to the effectiveness of the present invention. The nature of the movement is predetermined by careful selection of the properties of the support beam so that the movement is both predictable and reproducible.

For example, the support beam 11 may be made predictably flexible, rigid, or even internally actuated in order to impart the desired type of movement according to a particular patient's requirements.

The predetermined movement may be as a result of the material from which the support beam 11 is made, for example a flexible layered composite may be used which has a predetermined range of deflection.

Alternatively, or in addition, the predetermined movement may be as a result of the shape of the support beam 11. Many possible cross-sectional shapes for the support beam 11 may be envisaged, for example circular, elliptical, octagonal etc (see FIG. 5, in which suggested alternatives are indicated). Each shape of support beam imparts different types of relative movement, the surgeon being able to select a fixator having a cross-section appropriate to the patient's particular needs.

Alternatively, or in addition, the predetermined movement may be generated by or contributed to by one or more actuators embedded within the support beam 11. Other transducers may also be incorporated into the support beam 11, for example sensors for monitoring the predetermined movement or other physical property.

In some circumstances it is desirable to prevent relative movement of the respective ends of a fractured bone (for example in the treatment of hypertrophic non-union) and it is possible to have a rigid support beam in order to achieve this. The support beam 11 may be made from a memory alloy which is usually flexible but which, upon application of heat for example, may become rigid so as to prevent relative movement of the bone ends in order to treat such conditions. Consequently, the term “predetermined relative movement” encompasses the possibility of “no relative movement”.

The bone screw locating sleeves 20 each have a bore therethrough, through which a bone screw or the like can be inserted. In one embodiment, the bore of at least one bone screw locating sleeve 20 may be angled so that a bone screw inserted therein is directed towards a specific position with respect to the other bone screws.

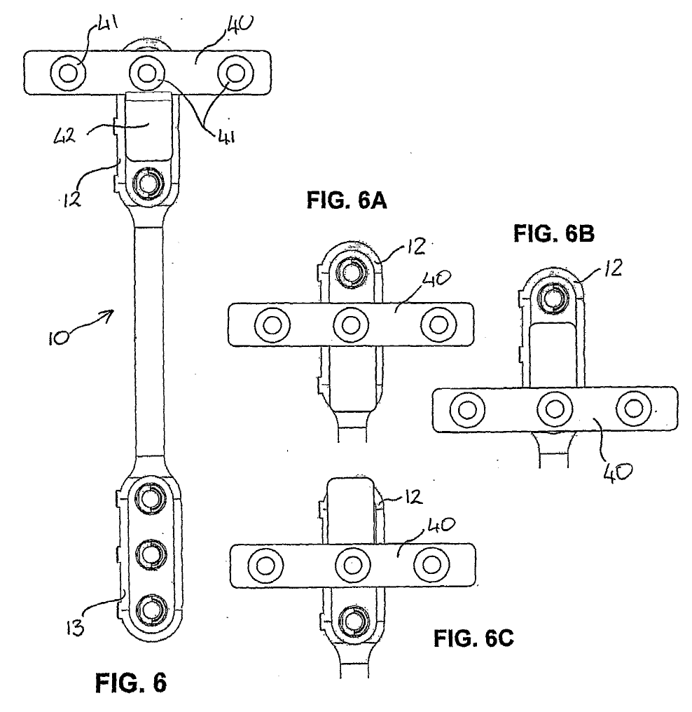

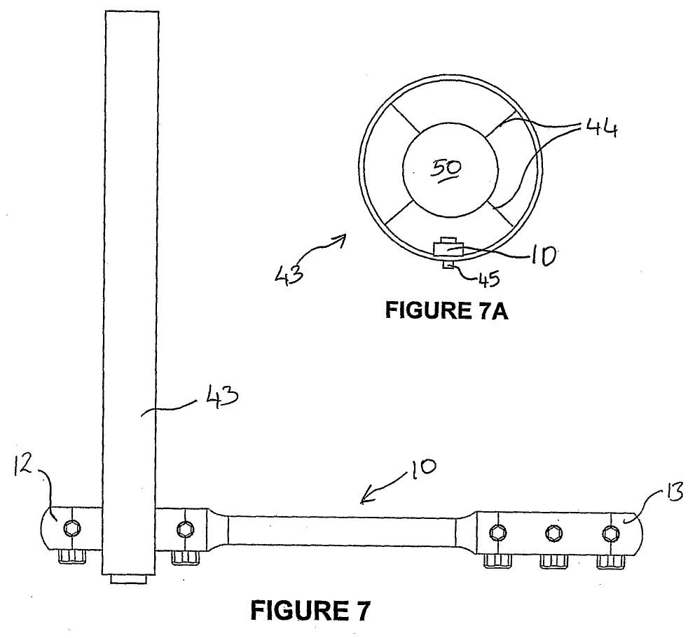

Hybridisation components such as ring fixators or T-bar attachments can be used readily with the fixator of the present invention and may by useful when fixating near a knee joint, for example. Examples of hybridisation components are illustrated in FIGS. 6-7A.

FIG. 6 is a top view of a fixator in which a T-bar attachment 40 has been attached to one end 12 of the fixator 10. The T-bar attachment enables bone screws to be fixed in an orientation that is perpendicular to the longitudinal axis of the fixator 10. In use, the bone screws (not illustrated) are located in apertures 41 of the T-bar attachment 40. The short leg 42 of the T-bar attachment is fixed into the underlying aperture 14 of the fixator so that the T-bar attachment and fixator cannot move with respect to one another. As illustrated in FIGS. 6A-6C, various alternative configurations are possible, according to the patient's needs. In all cases, a further bone screw or pin can optionally be inserted into the unused aperture in the fixator end 12, in addition to the three bone screws/pins in the T-bar attachment apertures 41.

FIG. 7 is a side view of a fixator 10 to which is attached a ring attachment 43. A top view of the ring attachment 43 is shown in FIG. 7A. The ring attachment 43 is attached to one end 12 of the fixator 10 by means of fixings 45 through any one of the apertures 14. Tension wires 44 and the bone 50 to which the fixator and ring attachment are attached are illustrated in FIG. 7A.

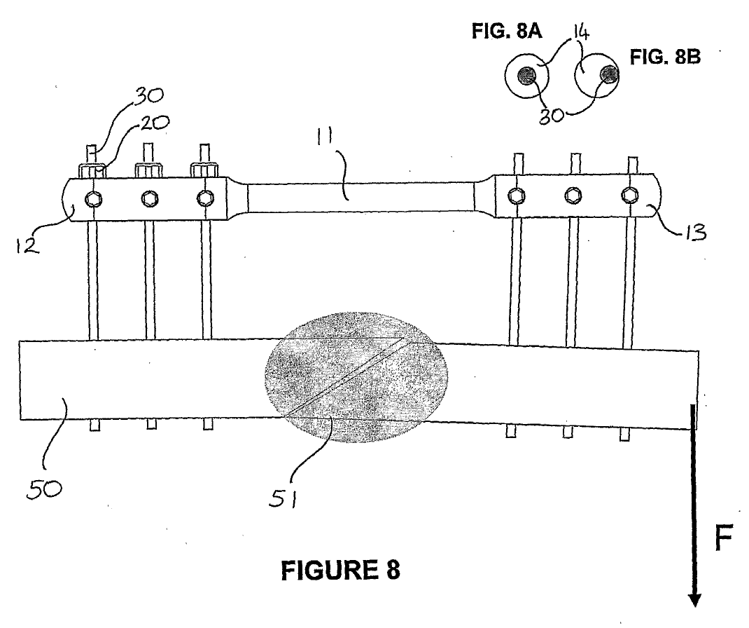

Referring to FIG. 8, the apertures 14 in the enlarged heads of the fixator may be used as a healing indicator with which the stiffness of the bone to which the fixator is attached can be tested to determine whether it is sufficiently healed. This is done by removing the bone screw locating sleeves 20 from one end of the fixator (hereafter called the “loose end”, 13), leaving the other end (the “fixed end”, 12) of the fixator properly attached to the bone screws (with bone screw locating sleeves still in place). The support beam 11 is then deflected by hand or by ambulation by as much as the bone 50 (to which the “fixed end” is still attached) will allow. During this deflection, the bone screw 30 located within aperture 14 at the “loose end” will move within the aperture 14. FIGS. 8A and 8B show the unloaded and loaded conditions respectively. It may be deemed that, if the possible deflection is sufficient to cause the bone screw to touch the periphery of aperture 14 at the “loose end” (as shown in FIG. 8B), the bone 50 is not yet sufficiently stiff to be properly healed. The build-up of callus at the junction between the bone fragments is indicated by reference numeral 51 in FIG. 8.

The fixator described herein is preferably disposable. The simplicity of the fixings means that the fixator can be easily removed and replaced on the bone screws, for example, to allow testing of the degree of healing or to heat a memory alloy fixator to make it rigid (see above).

The precision of the placing of the bone screws, selection of the shape and/or materials for the support beam etc mean that a range of fixators can be made, each capable of different (but predictable and reproducible) predetermined movement so that each fixator can be selected according to a particular patient's needs in order to minimise healing time. In addition, the fixator is much lighter, smaller and easier to fit and remove than prior art fixators so that patient discomfort is reduced and theatre time minimised.

As an example of how the fixator can be used in practice, here follows a description of how the fixator can be applied to a fractured tibia once the fracture has been reduced, for example using the STAFFORDSHIRE ORTHOPAEDIC REDUCTION MACHINE (described in PCT/GB98/00884).

It is necessary that fracture reduction is complete (i.e. no further reduction required) when the fixator is to be applied, if the fixator of the present invention is to be used. The STAFFORDSHIRE ORTHOPAEDIC REDUCTION MACHINE (described in PCT/GB98/00884) provides reduction of suitable accuracy.

After reduction, it is expected that the respective ends of the fractured bone will be held by means of bone screws associated with the reduction machine. Importantly, these reduction machine bone screws are not necessarily the bone screws to which the fixator will be applied.

There are six bone screws 30 for the fixator 10, each having an outside diameter of 6 mm, and which are inserted into the antero-medial surface of the tibia. Three screws are placed in the proximal fragment and three in the distal fragment.

Uniquely, the fixator 10 also acts as the drilling guide for the bone screw/pin sites. The normal operative technique now follows where suitable drill guides are used in conjunction with apertures 14 to pre-drill the six holes for the bone screws/pins.

What is claimed is:

1. Bone fixator for use in the treatment of a fractured bone comprising a support beam having means for attaching each end thereof to the respective ends of a fractured bone, the support beam being configured so as to permit predetermined relative movement between the respective ends of the support beam and thereby transmitting said relative movement to the respective ends of the fractured bone.

2. Bone fixator as claimed in claim 1 wherein said support beam is a one-piece support beam with no articulated joints therein.

3. Bone fixator as claimed in claim 1 wherein said means for attaching each end of the support beam to the respective ends of a fractured bone comprise apertures for receiving therein bone pins, bone screws, wires or the like.

4. Bone fixator as claimed in claim 3 wherein said apertures can be used as location guides for the drilling of holes for the bone pins, bone screws, wires or the like.

5. Bone fixator as claimed in claim 3 further comprising bone screw sleeves, each of which locate in one of said apertures, each bone screw sleeve having a bore therethrough for receiving therein bone pins, bone screws, wires or the like.

6. Bone fixator as claimed in claim 5 wherein one or more of said bone screw sleeves has an angled bore therethrough.

7. Bone fixator as claimed in claim 3 wherein at least one of said apertures can be used as a healing indicator by observing the possible positions of a bone pin or bone screw located therein relative to the periphery of said aperture.

8. Bone fixator as claimed in claim 1 wherein said bone fixator is disposable.

9. Bone fixator as claimed in claim 1 wherein the predetermined relative movement between the respective ends of the support beam is a property of the material of the support beam.

10. Bone fixator as claimed in claim 1 wherein the predetermined relative movement between the respective ends of the support beam is a property of the shape of the support beam.

11. Bone fixator as claimed in claim 1 wherein said support beam is made from a titanium alloy.

12. Bone fixator as claimed in claim 1 wherein said support beam is made from a layered composite material.

13. Bone fixator as claimed in claim 1 wherein said support beam includes one or more actuators which, in use, generate or contribute to said predetermined movement.

14. Bone fixator as claimed in claim 1 wherein said fixator is adapted to receive three bone pins, bone screws, wires or the like at each end thereof.

15. Bone fixator as claimed in claim 1 adapted to support a hybridisation component such as a T-bar attachment or ring attachment at one end thereof.

16. (canceled)

17. A bone fixation system comprising
a) a bone fixator for use in the treatment of a fractured bone comprising a support beam having means for attaching each end thereof to the respective ends of a fractured bone the support beam being configured so as to permit predetermined relative movement between the respective ends of the support beam and thereby transmitting said relative movement to the respective ends of the fractured bone; and
b) bone pins, bone screws, wires or the like for attaching the fixator to the respective ends of a fractured bone.

18. The bone fixation system as claimed in claim 17 further comprising a hybridisation component comprising a T-bar attachment or ring attachment.

19. The bone fixation system as claimed in claim 17 further comprising drilling guides for locating a drill with respect to said fixator.

20. (canceled)
